(19) United States
(12) Patent Application Publication
(10) Pub. No.: US 2015/0134034 A1
(43) Pub. Date: May 14, 2015

(54) VASODILATION SLEEVE AND METHOD OF VASODILATING A LIMB

(71) Applicant: Encompass Group, LLC, McDonough, GA (US)

(72) Inventor: Kelley Terrell, Ferris, TX (US)

(73) Assignee: Encompass Group, LLC, McDonough, GA (US)

(21) Appl. No.: 14/075,562

(22) Filed: Nov. 8, 2013

(51) Int. Cl. A61F 7/02

(52) U.S. Cl. CPC A61F 7/02; A61F 2007/0225; A61F 2007/0266; A61F 2007/0282
USPC 607/108

(57) ABSTRACT

A vasodilation sleeve (10) is disclosed having a tubular middle section (11) having on opening (12) at each end to which a stretchable cuff (13). The tubular middle section (11) is made of a soft, flexible heat reflective material which retains a patient's body heat. The sleeve (10) is placed upon a patient's limb over the site of a vascular puncture. The patient's body heat is trapped within the sleeve (10) causing a warming of the skin, the heat reflective material aids in quickly warming the limb which causes the underlying veins to dilate.

### TECHNICAL FIELD

This invention relates generally to a sleeve worn upon a patient's limb to promote vasodilation arid a method of promoting vasodilation in a patient's limb.

### BACKGROUND OF THE INVENTION

People oftentimes are required to inject or extract fluids into and out of the veins, which is accomplished through venipuncture. With some people the veins are not readily visible or accessible. As such, it may be difficult to find a vein and insert a hypodermic needle or other type of vein puncturing device into the vein, To promote vasodilation a tourniquet is placed about a patient's limb to constrict the blood flow through the veins and thereby cause dilation of the veins, which is commonly referred to as vasodilation.

Vasodilation is difficult to accomplish when a patient is obese, cold, has degraded veins, or has other medical condition effecting the veins. These problems can result in multiple attempts to achieve a proper venipuncture or the inability to accomplish this task all together. Devices have been designed to provide an ancillary heat source to the limb to cause vasodilation, such as that shown in U.S. Patent Application Publication Number 2012/0078223 A1. However, it would be desirous to achieve vasodilation without having to resort to such external heat sources.

Accordingly, it is seen that a need remains for a device that can aid in achieving vasodilation in an effective manner without an ancillary heat source. It is to the provision of such therefore that the present invention is primarily directed.

### SUMMARY OF THE INVENTION

In a preferred form of the invention a vasodilation sleeve comprises an elongated, tubular sleeve having two oppositely disposed open ends having stretchable cuffs. The elongated, tubular sleeve is made of a heat reflective material.

In another preferred form of the invention a method of inducing vasodilation comprises the steps of positioning a heat reflective sleeve about the limb of a person, maintaining the heat reflective sleeve in position upon the limb of a person for a sufficient time to allow the limbs body heat to reflect off the heat reflective sleeve and build to a level which causes vasodilation of a vein located within a region of the limb covered by the heat reflective sleeve, and moving the heat reflective sleeve to a position exposing the vasodilated vein of the limb.

### BRIEF DESCRIPTION OF THE DRAWING

FIG. 1 is a perspective view of a vasodilation sleeve embodying principles of the invention in a preferred form.

FIG. 2 is a cross-sectional view of the vasodilation sleeve of FIG. 1.

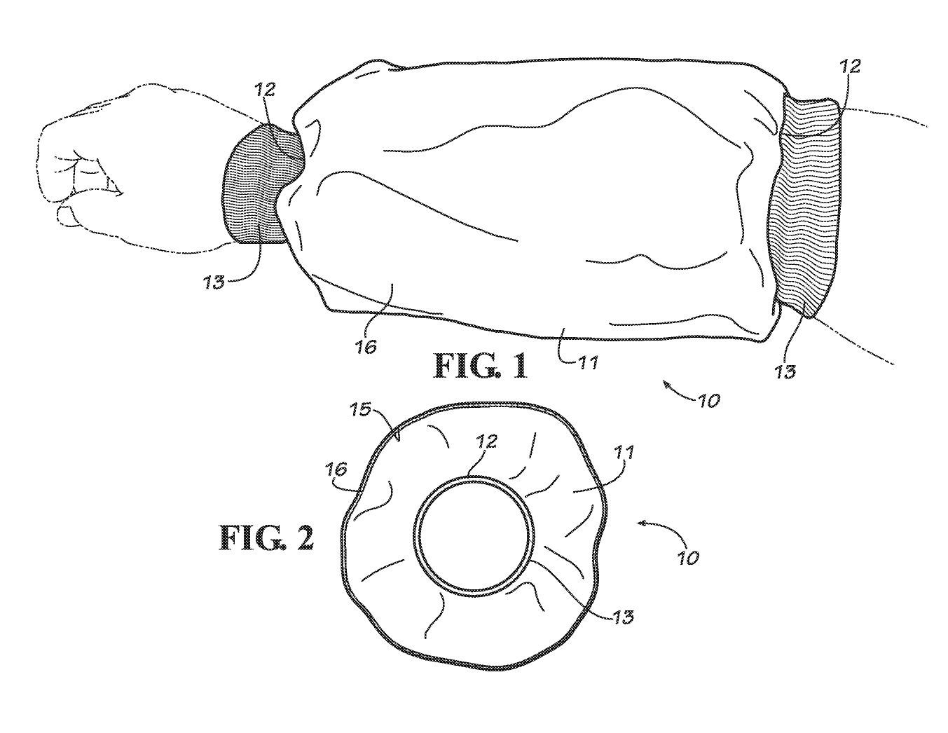

### DETAILED DESCRIPTION

With reference next to the drawings, there is shown a vasodilation sleeve 10 in a preferred form of the invention, The sleeve 10 has an elongated, generally cylindrical or tubular middle section 11 having on opening 12 at each end to which an elastic or stretchable knit cuff 13 is coupled.

The tubular middle section 11 is of a length and circumference to fit upon a large portion of a patient's limb, such as an arm or leg. The tubular middle section 11 is preferably made of a soft, flexible material which retains a patient's body heat, such as the heat reflective metalized fabric material such as that sold by Encompass Group, LLC of McDonough, Ga. under the tradename THERMOFLECT. The metalized fabric material has an interior facing layer or surface 15 which is a lofted billow spunbond thermoplastic non-woven material for comfort of the patient and an exterior facing layer or surface 16 which is a vaporized aluminum layer which is coated on both sides with a clear polyethylene layer to greatly reflect body heat.

The cuff 12 is made of a knitted cotton to provide the stretch and elasticity required for the cuff to fit over a patient's hand or foot and still retain its shape and snug fit about a patient's limb to restrict airflow therebetween which would allow the release of body heat.

In use, the sleeve 10 is placed upon a patient's limb so that the site of the vascular puncture is located beneath the sleeve middle section, and preferably at approximately at the center of the sleeve middle section. With the sleeve 10 in this position that cuffs 13 should fit snuggly about the limb of the patient to restrict the loss of body heat through the cuff area. Once the sleeve 10 is in place upon a limb, the patient's body heat is reflected by the sleeve heat reflective fabric material and is therefore trapped within the sleeve 10, thereby causing a warming of the skin The elastic cuffs 13 ensure that the body heat does not escape through the end openings 12. Within a short span of time of approximately 5 to 8 minutes, the warming of the skin causes the underlying veins to dilate, i.e., vasodilate, thereby making the veins more pronounced and accessible for vascular puncture. Once the veins have been dilated, the medical professional can move the sleeve up or down along the limb, or remove it completely from the limb, and puncture the vein with the proper device.

It should be understood that as an alternative in the cuffs 13 shown in the preferred form of the invention, the openings 12 may be fitted with an elastic member or other type of gathering means to partially close or gather the sleeve at the openings to provide a snug fit of the openings over a patient's limb to prevent the escape of body heat through the openings However, for the comfort of a patient, it is believed that a cuff provides the best fit and heat sealing capabilities while still providing the greatest degree of comfort to the wearer.

It should be understood that the term heat reflective material or the like as used herein is intended to represent a material which is specifically designed to reflect human body heat or endogenous radiant heat to a substantially high degree. The term heat reflective material, or the like, is not intended to represent any and all materials that reflect only a small portion of the body's heat, as substantially any material is capable of reflecting at least some degree of body heat from its existence alone, such as a knit, woven, nonwoven, extruded cotton, wool, polymer, paper of other similar material as well as materials designed to allow a substantial transfer of heat or the flow of gases therethrough.

It thus is seen that a vasodilation sleeve and a method of vasodilating a limb is now provided that does not require an ancillary heat source. Although the vasodilation sleeve and method has been illustrated and described in its preferred form, it should be understood that many modifications, additions and deletions may be made to that specific form without departure from the spirit and scope of the invention as set forth in the following claims.

1. A vasodilation sleeve comprising:
an elongated, tubular sleeve having two oppositely disposed open ends, said elongated, tubular sleeve being made of a heat reflective material; and
an stretchable cuff coupled to each said two oppositely disposed open ends of said elongated, tubular sleeve and configured to fit snugly about a limb of a person.

2. The vasodilation sleeve of claim 1 wherein said heat reflective material is a metalized fabric material.

3. The vasodilation sleeve of claim 2 wherein said metalized fabric material has a metallic exterior surface and a spunbond interior surface.

4. A vasodilation sleeve comprising:
a tubular, central portion having a first opening at a first end and a second opening at a second end;
first gathering means for partially gathering said first opening to fit snugly about a person's limb to restrict the airflow through said first opening, and
second gathering means for partially gathering said second opening to fit snugly about a person's limb to restrict the airflow through said second opening.

5. The vasodilation sleeve of claim 4 wherein said tubular, central portion is made of a heat reflective material.

6. The vasodilation sleeve of claim 5 wherein said heat reflective material is made of a metalized fabric material.

7. The vasodilation sleeve of claim 6 wherein said metalized fabric material has a metallic exterior surface and a spunbond interior surface.

8. A method of inducing vasodilation comprising the steps of:
(A) positioning a heat reflective sleeve about the limb of a person;
(B) maintaing the heat reflective sleeve in position upon the limb of a person for a sufficient time to allow the limbs body heat to reflect off the heat reflective sleeve and build to a level which causes vasodilation of a vein located within a region of the limb covered by the heat reflective sleeve, and
(C) moving the heat reflective sleeve to a position exposing the vasodilated vein of the limb.

9. The method of claim 8 wherein said heat reflective sleeve is made of a metalized fabric material.

10. The vasodilation sleeve of claim 9 wherein said metalized fabric material has a metallic exterior surface and a spunbond interior surface.

11. The method of claim 8 wherein said heat reflective sleeve includes a heat reflective central portion having two oppositely disposed openings and an stretchable cuff coupled to each of the two oppositely disposed openings.
